(19) United States
(12) Plant Patent Application Publication
(10) Pub. No.: US 2016/0324057 P1
(43) Pub. Date: Nov. 3, 2016

(54) Delphinium plant named 'Coadelbol'

(50) ;

(71) Applicant: Anthony Paul Coakley, Glasgow (GB)

(72) Inventor: Anthony Paul Coakley, Glasgow (GB)

(21) Appl. No.: 14/545,379

(22) Filed: Apr. 28, 2015

(51) Int. Cl. A01H 5/00

(52) U.S. Cl. PLT/423

(57) ABSTRACT

‘Coadelbol’ is a distinctive variety of Delphinium which is characterized by the combination of flowers with a large quantity of tepals and petaloids that exhibit a unique color combination of dark purple, blue and violet with pale yellow-green centers, an upright plant habit, a vigorous rate of growth, and the stability and uniformity of these traits through successive cycles of asexual propagation. The new cultivar produces a strong main flowering stem as well as a free and continuous bloom habit from early through late summer.

### LATIN NAME OF THE GENUS AND SPECIES

The Latin name of the genus and species of the novel variety disclosed herein is Delphinium hybrid.

### VARIETY DENOMINATION

The inventive variety of Delphinium disclosed herein has been given the variety denomination ‘Coadelbol’.

### BACKGROUND OF THE INVENTION

The present invention relates to a new and distinct cultivar of Delphinium, which has been given the variety denomination of ‘Coadelbol’. Its market class is PLT/423. The genus Delphinium is commonly known as a member of the Ranunculaceae family and may also be referred to as Larkspur. Delphinium plants bear inflorescences of a showy spike or racemes that may sometimes be paniculate and contain few to many flowers on erect and sometimes branching stems. The genus Delphinium is unusual in that all three primary colors are represented in different species. The tall hybrids with their long spikes are available in a wide range of colors including purple, mauve, pink, white, and cream as well as varying shades of blue. In Europe, they are grown as perennials but are treated as annuals in California and regions of similar climate.

### Parentage

The Delphinium variety ‘Coadelbol’ is the result of a planned breeding program carried out in Glasgow, Scotland. ‘Coadelbol’ is a seedling selection which resulted from the controlled pollination of two proprietary Delphinium hybrid breeding accessions (unnamed, uncommercialized and undistributed) developed and owned by the inventor. The initial selection of the cultivar now known as ‘Coadelbol’ was made in August of 2008. At that time it was determined that said cultivar exhibited certain distinguishing characteristics that would prove favorable for commercial production and marketability.

### Asexual Reproduction

‘Coadelbol’ was first asexually propagated by tissue culture in 2012 at a tissue culture laboratory in Roelofarendsveen, The Netherlands under the control of the inventor. The distinctive characteristics of the inventive ‘Coadelbol’ cultivar are stable from generation to generation; clones of the cultivar produced by tissue culture maintain the distinguishing characteristics of the original plant.

### SUMMARY OF THE INVENTION

The following characteristics have been repeatedly observed and represent the distinguishing characteristics of the new Delphinium cultivar ‘Coadelbol’. These traits in combination distinguish ‘Coadelbol’ as a new and distinct cultivar.

1. Delphinium ‘Coadelbol’ exhibits a floret with a high tepal and petaloid count.

2. Delphinium ‘Coadelbol’ exhibits a unique color combination within the floret.

3. Delphinium ‘Coadelbol’ exhibits an upright growth habit.

4. Delphinium ‘Coadelbol’ exhibits a high degree of plant vigor.

5. Delphinium ‘Coadelbol’ exhibits a primary inflorescence with a strong peduncle.

6. Delphinium ‘Coadelbol’ exhibits a freely and continuous bloom habit.

### BRIEF DESCRIPTION OF THE FIGURES

FIG. 1 shows, as nearly true as it is reasonably possible to make the same in color illustrations of this type, the foliage of the new cultivar, ‘Coadelbol’. The plant shown was grown outdoors in Glasgow, Scotland using conventional production practices typically employed for producing Delphinium plants. No disease or pest measures were taken in producing this plant.

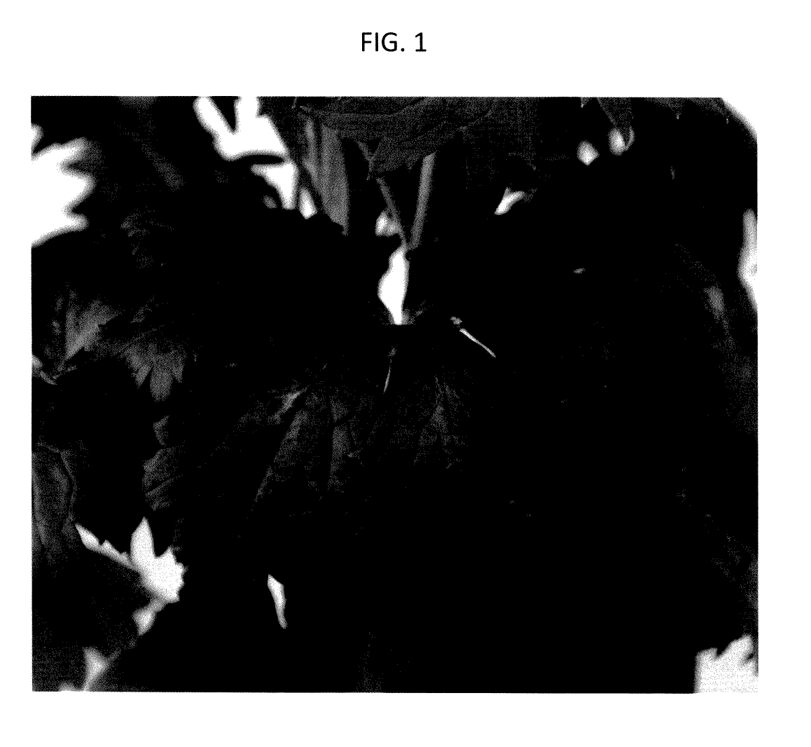

FIG. 2 shows, as nearly true as it is reasonably possible to make the same in color illustrations of this type, the inflorescence of ‘Coadelbol’.

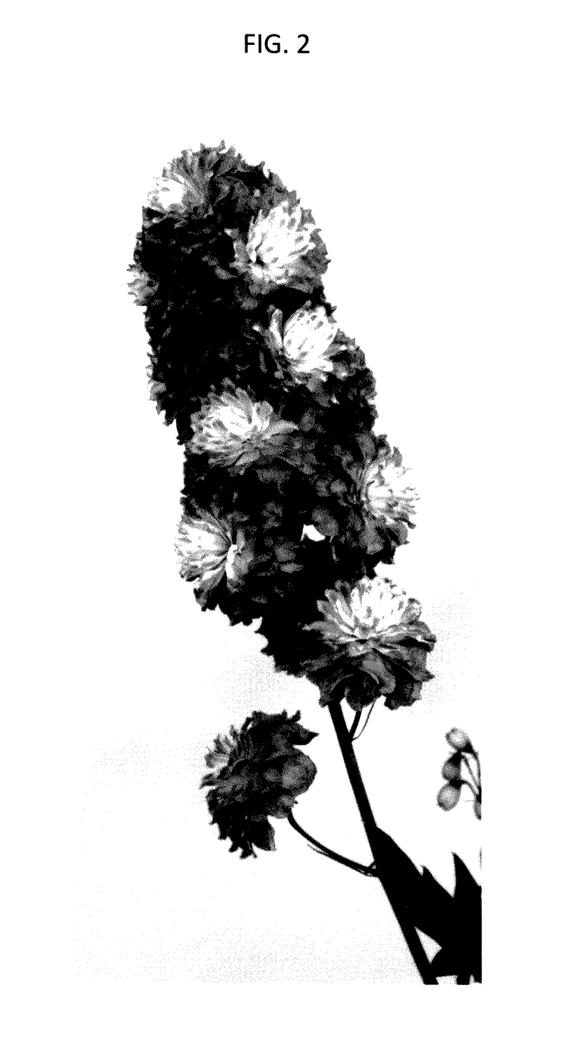

### BOTANICAL DESCRIPTION OF THE PLANT

The following is a detailed botanical description of a new and distinct variety of Delphinium known as ‘Coadelbol’, based upon observations of a 9 month old plant grown outdoors in Glasgow, Scotland in a one liter nursery pot from winter to summer, using conventional production practices typically employed for producing Delphinium plants. No disease or pest measures were taken in producing this plant. Observation data was recorded in August of 2014.

Those skilled in the art will appreciate that certain characteristics will vary with older or, conversely, with younger plants. ‘Coadelbol’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 2007 edition.

A botanical description of ‘Coadelbol’ and comparisons with other varieties of Delphinium are provided below.
Technical description of the variety:
Plant description:
   Growth rate.—Moderate.
   Growth habit.—Upright perennial flowering plant with flowers arranged on terminal racemes; numerous racemes per plant.
   Height.—Approximately 95.0 cm.
   Width.—Approximately 60.0 cm.
   Environmental tolerances.—Tolerates temperatures as low as 5 approximately minus 20 degrees Celsius (twenty degrees below freezing); upper temperature tolerance is unknown. No tolerance for drought.
   Disease and pest resistance.—Plants of the claimed plant have not been noted to be resistant to pathogens and pests common to Delphinium.
   Propagation.—Tissue culture.
   Time to produce a rooted cutting.—Approximately 4 to 5 weeks to produce a rooted cutting.
Root system: Fine, fibrous; freely branched and moderately dense.
Stem:
   Quantity of lateral branches.—Approximately 3.
   Stem cross-section.—Slightly to moderately quadrangulate
   Stem length.—Approximately 50 cm, excluding the inflorescence.
   Stem width.—Approximately 0.1 cm.
   Stem color.—Near RHS 143A.
   Stem texture.—Glabrous; matte, due to a very thin waxy layer, the color of which is near RHS 191D.
   Internode length.—Approximately 1.9 cm.
Foliage:
   Leaf arrangement.—Alternate.
   Leaf attachment.—Petiolate.
   Quantity of leaves per plant.—Approximately 27 per main branch.
   Leaf length.—Average of 19 cm in length.
   Leaf width.—Average of 22 cm in width.
   Leaf shape.—Pedately lobed.
   Leaf apex.—Acute.
   Leaf base.—Hastate to strongly hastate.
   Leaf margin.—Biserrate.
   Texture (adaxial surface).—Glabrous; slightly glossy.
   Pubescence (adaxial surface).—Absent.
   Young leaf color (adaxial surface).—Near RHS 143A to 143B.
   Young leaf color (abaxial surface).—Near RHS 143C.
   Mature leaf color (adaxial surface).—Near RHS N137A but slightly darker.
   Mature leaf color (abaxial surface).—Near RHS 147B.
   Venation.—Laciniate.
   Vein color (adaxial surface).—Near RHS 144A.
   Vein color (abaxial surface).—Near RHS 144A.
   Petiole.—Approximately 11.9 cm long and 0.45 cm wide; color is near 144A.
Inflorescence:
   Inflorescence type.—Terminal raceme.
   Inflorescence dimensions.—Approximately 44.1 cm long, as measured from the lowest flower to the apex of the inflorescence, and 9.1 cm wide.
   Bracts.—Quantity — Two bracts are present at the base of each pedicel. Shape — Linear. Length — 1.4 cm, on average. Width — 0.1 cm, on average. Apex — Narrowly acute. Margin — Entire. Color (adaxial and abaxial surfaces) — Near RHS 143A.
Bud:
   Flower bud shape.—Obovate.
   Flower bud length.—1.3 cm, excluding the spur.
   Flower bud diameter.—Averaging 0.9 cm.
   Bud color.—Near RHS 94B to 94C, tinged near RHS 144B to 144C, with lower side colored near RHS 149D.
Flower
   Flowering quantity.—About 90 buds and 98 fully opened flowers per lateral branch, at one time.
   Natural flowering season.—Midsummer to late summer.
   Flower longevity on plant.—Approximately three weeks.
   Pedicel.—Length — Approximately 3.1 cm. Diameter — Approximately 1.5 mm. Texture — Smooth; glabrous. Color — Near RHS 143B and 144B.
   Perianth.—Consisting of a spur, a single whorl of tepals and multiple whorls petaloids; the approximate diameter is 4.8 cm and the approximate depth is 2.7 cm, excluding the spur. Aspect of corolla is upward to slightly pendulous. Flowers are persistent. Tepal Description — Arrangement — Rotate. Quantity — Five. Length — Approximately 2.5 cm. Width — Two lower tepals average 1.6 cm and three upper tepals average 2.0 cm Shape — Obovate. Margin — Entire, undulate. Apex — Broad acute. Texture — Glabrous, slightly glossy and slightly velvety. Aspect — Slightly curved backwards. Color, When Opening (adaxial surface) — Near RHS 99B to 99C; base and central stripe tinged near RHS 146C. Color, When Opening (abaxial surface) — Near RHS 98A and 99D; base and central stripe tinged near RHS 146B and 146C. Color, Fully Opened (adaxial surface) — Near RHS 99B; base and central stripe tinged near RHS 146C. Color, Fully Opened (abaxial surface) — Near RHS 98A and 99D; base and central stripe tinged near RHS 146B and 146C. Petaloid Description Quantity — 15. Arrangement — Rotate. Length — Average: 2.1 cm. Width — Average: 1.2 cm. Shape — Obovate. Margin — Entire, undulate. Apex — Broad acute. Texture — Glabrous, slightly glossy, and slightly velvety. Aspect — Very slightly curved backwards. Color, When Opening (adaxial surface) — Near RHS NN155D, base and central stripe near RHS 145D, tip near RHS 94A. Color, When Opening (abaxial surface) — Near RHS NN155D, base and central stripe near RHS 145D, tip near RHS 94A. Color, Fully Opened (adaxial surface) — Near RHS 95C and 85A, base and central stripe near RHS 145B to 145D. Those petaloids near the center of the perianth are colored near RHS 157D, base, central stripe and tip near 145B to 145D. Color, Fully Opened (abaxial surface) — Near RHS 95C and 85A, base and central stripe near RHS 145B to 145D. Those petaloids near the center of the perianth are colored near RHS 157D, base, central stripe and tip near 145B to 145D
   Spur.—Shape — Oblong. Quantity per flower — 1. Length — Approximately 0.6 cm. Width — Approximately 0.3 cm. Margin — Entire. Texture — Glabrous, bullate. Color — Near RHS 146C, heavily tinged near RHS 94B to 94C.
Reproductive organs:
   Stamens.—No stamens are present; all stamens are transformed into petaloids.
   Pistil.—No pistils are present, all pistils are transformed into petaloids.
Seed and fruit: None observed.
Comparisons with the parents: Plants of the new cultivar ‘Coadelbol’ are similar to plants of the seed parent variety, an unnamed Delphinium hybrid, in most horticultural characteristics. However, flowers of the new variety have a higher tepal count with a stronger, unique and more consistent color. Plants of the new cultivar ‘Coadelbol’ are similar to plants of the pollen parent variety, an unnamed Delphinium hybrid, in most horticultural characteristics. However, flowers of the new variety have a higher tepal count with a stronger, unique and more consistent color.
Comparisons with similar Delphinium cultivars known to the inventor: The inventor is unaware of similar commercial varieties for comparison to ‘Coadelbol’. The strong flowering stems, unique flower coloration and increased tepal count make the new cultivar unlike other Delphiniums in the trade.

That which is claimed is:

1. A new and distinct variety of Delphinium plant named ‘Coadelbol’, substantially as described and illustrated herein.
